(19) United States
(12) Patent Application Publication
(10) Pub. No.: US 2009/0304831 A1
(43) Pub. Date: Dec. 10, 2009

(54) Laxative Composition and Method

(76) Inventors: Ken CLEMENT, (US); Marvin A. Heuer, (US); William Ha, (US); Megan Thomas, (US); Erin Taraborrelli, (US); Krista Rutherford, (US)

Correspondence Address: IOVATE HEALTH SCIENCE RESEARCH INC., 381 North Service Road West, Oakville, ON, L6M 0H4, CA

(21) Appl. No.: 12/477,566

(22) Filed: Jun. 3, 2009

Related U.S. Application Data
(60) Provisional application No. 61/058,400, filed on Jun. 3, 2008.

(51) Int. Cl. A61K 36/482; A61K 36/72; A61K 36/708; A61P 1/00

(52) U.S. Cl. 424/757; 424/725

(57) ABSTRACT

An improved laxative composition is provided having efficacy with reduced negative side-effects. The laxative composition is also provided with agents that promote healthy intestinal and liver function, reduce carbohydrate absorption and have antioxidant capacity. A method is also provided for reducing the negative side-effects associated with Anthranoid-based laxative compositions. A method is further provided for increasing the efficacy and/or duration of effect of Anthranoid-based laxative compositions.

### RELATED APPLICATIONS

The application is related to and claims benefit of priority to Applicant's U.S. Provisional Patent Application Ser. No. 61/058,400 entitled “Improved Laxative Composition and Method,” filed Jun. 3, 2008, the disclosure of which is hereby fully incorporated by reference.

### FIELD OF THE INVENTION

The invention relates generally to improved laxative compositions and methods. More specifically, the invention provides improved laxative compositions and methods by combining Anthranoid laxatives with agents that slow digestion, intestinal transit time, or the rate of food absorption.

### BACKGROUND OF THE INVENTION

Anthranoids are a group of chemical compounds of natural origin and belong to one of three groups: Anthrones, Anthraquinones and Dianthrones. Plants containing Anthranoids are generally considered herbal laxatives and have both traditional and contemporary applications. The Anthranoids from plants are typically bound by sugar moieties via a chemical linkage that is resistant to breakdown in the stomach and small intestine. Once in the large intestine, bacteria metabolize the inactive Anthranoids into active forms, some of which may be absorbed where they undergo further metabolism and subsequent excretion.

A potential problem sometimes associated with the use, and often with the overuse or abuse, of Anthranoid laxatives is the occurrence of diarrhea and cramping resulting from excessive epithelial cell damage combined with, or related to, decreased fluid absorption, and increased secretion.

### SUMMARY OF THE INVENTION

The present invention relates to improved laxative compositions combining at least one Anthranoid laxative with at least one soluble fiber.

An additional aspect of the present invention relates to methods of reducing the negative side-effects of Anthranoid laxatives by administering a composition that combines at least one Anthranoid laxative with at least one soluble fiber.

A further aspect of the present invention relates to methods of increasing the efficacy and/or duration of effect of Anthranoid laxatives by administering a composition that combines at least one Anthranoid laxative with at least one soluble fiber.

According to another aspect, the present invention relates to laxative compositions comprising agents which promote healthy intestinal and liver function.

According to another aspect, the present invention relates to laxative compositions comprising agents which reduce carbohydrate absorption.

According to yet another aspect, the present invention relates to laxative compositions comprising antioxidants.

### DETAILED DESCRIPTION OF THE INVENTION

As used herein, the term “Anthranoid laxative” is understood to describe any composition intended for use as a laxative that contains any chemical substance belonging to the Anthranoid family. The Anthranoid may be present in the form of a raw plant, a plant extract, purified form, or a synthetic form. Sources of Anthranoids include, but are not limited to, Senna (Cassia angustifolia), Cascara (Rhamnus purshiana) and Rhubarb (Rheum palmatium).

As used herein, the term “soluble fiber” is understood to describe any of the class of carbohydrate-type polymers that are frequently found in human diets and includes, but is not limited to, cellulose gums, pectins, galactomannans, glucomannans, beta-glucans and others.

Senna (Cassia angustifolia) is a laxative that contains Anthraquinone derivatives, or Sennosides, which are some of the most well-known Anthranoids. The efficacy of Senna as a laxative is related to the stimulation of intestinal motility and increased large intestinal transit via colonic smooth muscle contractions and its effects on the epithelial transport of water and electrolytes. Other plants traditionally used as laxatives, such as Cascara (Rhamnus purshiana) and Rhubarb (Rheum palmatium), also contain Anthranoids.

The laxative effects of Anthranoids are generally encountered 4-5 hours after ingestion. This is related to the time required for transport to the large intestine and metabolism. The effects of Anthranoid laxatives are dependent upon contact with the intestinal epithelium and are related to changes in the epithelial cells lining the large intestine that initiate signaling cascades.

Glucomannan is a soluble polysaccharide fiber of chains of glucose and mannose from the root of konjac (Amorphophallus konjac). Glucomannan has the highest viscosity of all known polysaccharides which is correlated with its ability to improve glycemic control and lipid profile by reducing total cholesterol and low-density lipoprotein levels, reduce associated risk factors such as hyperlipidemia and hypertension, and improve insulin resistance. Studies have supported glucomannan as an effective agent for improving glycemic control, serum lipids, and systolic blood pressure in high-risk type 2 diabetes patients. Glucomannan's effects may be related to its viscosity and gel-forming qualities and the subsequent slowing of the rate of digestion and food absorption. Other suitable soluble fibers include Guar gum, Xanthan gum, and a synthetic soluble fiber, Hydroxypropylmethyl cellulose.

While both Senna and Cascara have been widely and traditionally used as stimulant-laxatives there have been problems associated with their use, particularly diarrhea.

The inventors have found that combining an Anthranoid laxative such as Senna or Cascara, which is associated with an increase of intestinal motility, with a soluble fiber such as Glucomannan, which is associated with a slowing of food absorption, results in an efficacious laxative formulation with attenuated negative effects typically associated with Anthranoid laxatives.

Advantageously, additional embodiments of the present invention may combine at least one Anthranoid laxative and at least one soluble fiber with additional substances commonly known in the art to act as a laxative or in any way promotes laxative effects.

Furthermore, the laxative compositions of the present invention can further comprise one or more agents which promote healthy intestinal, liver function and reduce carbohydrate absorption. Moreover, the laxative compositions can further comprise one or more antioxidants.

Digestion of consumed food is a complex and important process involving many steps, processes and organs. Foods are broken down for absorption and use. Sugars from carbohydrates, amino acids from proteins, and fatty acids and glycerol from fats are transported through the intestinal wall into the bloodstream for use or processing.

The liver serves many functions in the body, including digestion, in number of ways. The liver secrets bile, which is stored in the gallbladder, to break down ingested fats. The liver also processes and/or filters substance entering the blood stream from ingested food and products of metabolism. The liver also stores and processes fats and carbohydrates and synthesizes certain proteins. Thus proper hepatic function aids the overall digestive process.

Silybum marianum, also known as milk thistle, has been traditionally used in treatment of liver diseases. Silymarin is one of the active ingredients which is extracted from the dried seeds of the plant and is believed to have hepato-protective properties. Therefore, it is herein understood that milk thistle, or an extract thereof, will act to benefit the digestive process by supporting liver function.

Dandelion (Taraxacum officinale) is a perennial weed that belongs to the genus Taraxacum and is a member of the Asteraceae family. It is widely distributed in the warmer temperate zones of the Northern Hemisphere, inhabiting fields, roadsides and ruderal sites. In North American aboriginal medicine, infusions and decoctions of the root and herb were applied to remedy kidney disease, dyspepsia, and heartburn. In Turkish popular medicine, this herb has been used as a laxative, a diuretic and a potent anti-diabetic agent. Dandelion promotes the flow of bile has also been shown to be effective as a diuretic, flushing excess water from the body. Research has shown that the aqueous extracts of the dandelion root and herb possess strong diuretic activities in animals. It is herein understood that Dandelion, or an extract thereof, will act to benefit the digestive process by supporting liver function and act as a diuretic.

Inulin is a type of naturally occurring fructose-containing oligosaccharide present in various fruits and vegetables such as onions, garlic, wheat, leeks, garlic, bananas, asparagus, and artichokes. Typically, inulin contains 2 to 150 fructose units which are linked by beta (2-1) glycosidic bond with a terminal glucose. Inulin is resistant to digestion in the upper gastrointestinal tract and is fermented by the colonic bacteria, promoting intestinal bacteria and possibly acting as a mild laxative. In the colon, inulin is metabolized into short-chain fatty acids-acetate, propionate, and butyrate, lactic acids, and gases (e.g., hydrogen sulfide, carbon dioxide, and methane). Inulin is also reported to possess anti-tumor, antimicrobial, hypolipidemic, hypoglycemic, and antiosteoporotic effects and enhance mineral absorption and balance. Inulin from roots of chicory and Jerusalem artichokes are marketed as nutritional supplements and functional foods. Nutritionally, it is considered a form of soluble fibre. It is herein understood that inulin will act to benefit digestion by enhancing nutrient absorption, by promoting intestinal bacteria, and by mild laxative activity.

Mulberry (Morus alba) is an edible plant used in Chinese medicine rich in flavonoids with antioxidant activity. Mulberry leaves have been shown to result in weight loss and reduced postprandial glucose increase, indicative of reduced carbohydrate absorption. Furthermore, a tea extract containing Mulberry has been shown to reduce carbohydrate absorption in humans. It is herein understood to be due to constituents of Mulberry that inhibit a-glucosidase.

Phaseolamin is a glycoprotein mainly found in beans and is most often extracted from white kidney beans (Phaseolus vulgaris) and is an inhibitor of alpha-amylase which breaks down starch. It is herein understood that inhibiting the breakdown of starch will reduce its absorption and contribute to weight loss. Clinical research has shown that phaseolamin reduces intestinal amylase activity and can reduce fat mass, while maintaining lean body mass.

As used herein, the term “derivative” refers to a compound, such as a salt, ester, or amine, which can readily supply a closely related biologically active compound, either upon administration or upon exposure to specific environmental conditions, such as pH, temperature, etc. For example, a “derivative” of anthraquinone can be a salt, ester or amine of anthraquinone, so long as the derivative can readily supply biologically active anthraquinone. For a given compound, the skilled person in the art will readily recognize and envisage those closely related compounds that should be considered “derivatives.”

Thus, for example, a derivative of a particular substance may comprise a form of that substance which has been modified through reaction. Other derivatives are forms of a given substance that are precursors of that substance which would give rise to that substance after modification. Reactions involved in the formation of derivatives include, but are not limited to: hydroxylation, esterification, amide formation and salt formation.

In addition to the foregoing, compositions of the present invention include formulations further comprising additional active ingredients and/or inactive ingredients, including solvents, diluents, suspension aids, thickening or emulsifying agents, sweeteners, flavorings, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and which may also be suitable for use in formulations of the present invention. Except insofar as any conventional carrier medium is incompatible with the ingredients of the invention, such as by producing any undesirable effect or otherwise interacting in a deleterious manner with any other ingredient(s) of the formulation, its use is contemplated to be within the scope of this invention.

### EXAMPLES

### Example 1

### Acute Administration of Glucomannan and Senna

In order to test the tolerance and efficacy of an improved laxative composition according to one embodiment of the present invention, two laxative samples were tested. In the first study, an acute dose of a test composition “A” (Sennosides 24 mg) was administered to 11 subjects. For comparison, in a second study 13 subjects were given composition “B” (Sennosides 24 mg, Glucomannan 2.25 g). In each case all subjects were asked to complete a questionnaire. A subset of 7 subjects was common to both tests.

### Questionnaire

### Composition A

1. Describe your normal level of regularity.
2. Describe your “normal” diet.
3. What time did you take the test product?
4. Did you consume the full dose? If not how many tablets did you take?
5. How soon after did you have an effect?
6. Describe the effect.
7. Did you have any negative side effects? Describe.
8. Was there greater volume than usual?
9. How long did the effect last?
10. Did you have reoccurring effects?
11. What was your overall feeling?
12. Was there any discomfort? If so describe.
13. Would you take this product again?

### Results—Composition A

### Subjects

N=11

### Normal Regularity

| | |
|---|---|
| 1-3 times per week: | 2 |
| 4-6 times per week: | 0 |
| Once daily: | 6 |
| Twice daily: | 2 |
| More than twice daily: | 0 |
| Not answered: | 1 |

### Time for Effect

| | |
|---|---|
| Immediate: | 0 |
| 1-4 hours: | 0 |
| 5-8 hours: | 4 |
| 9-12 hours: | 4 |
| Longer: | 2 |
| Not at all: | 1 |

### Side Effects

| | |
|---|---|
| None: | 4 |
| Cramping: | 6 |
| Nauseous: | 1 |
| Gas: | 2 |
| Diarrhea: | 0 |
| Other: | 3 |

### Full Dose Consumed

| | |
|---|---|
| Yes: | 11 |
| No: | 0 |

### Greater Volume

| | |
|---|---|
| Yes: | 6 |
| No: | 5 |

### Would Take Again

| | |
|---|---|
| Yes: | 8 |
| No: | 3 |

### Questionnaire

### Composition B

1. Describe your normal level of regularity.
2. Describe your “normal” diet.
3. What time did you take the test product?
4. Did you consume the full dose? If not how many tablets did you take?
5. How soon after did you have an effect?
6. Describe the effect.
7. Did you have any negative side effects? Describe.
8. Was there greater volume than usual?
9. How long did the effect last?
10. Did you have reoccurring effects?
11. What was your overall feeling?
12. Was there any discomfort? If so describe.
13. Would you take this product again?
14. Was there a difference between taking this combination or Senna on its own?

### Results—Composition B

### Subjects

N=13

### Normal Regularity

| | |
|---|---|
| 1-3 times per week: | 2 |
| 4-6 times per week: | 0 |
| Once daily: | 8 |
| Twice daily: | 3 |
| More than twice daily: | 0 |

### Time for Effect

| | |
|---|---|
| Immediate: | 0 |
| 1-4 hours: | 1 |
| 5-8 hours: | 3 |
| 9-12 hours: | 9 |
| Longer/Not at all: | 0 |

### Side Effects

| | |
|---|---|
| None: | 8 |
| Cramping: | 3 |
| Nauseous: | 0 |
| Gas: | 0 |
| Diarrhea: | 1 |
| Other: | 1 |

### Full Dose Consumed

| | |
|---|---|
| Yes: | 13 |
| No: | 0 |

### Greater Volume

| | |
|---|---|
| Yes: | 8 |
| No: | 5 |

### Would Take Again

| | |
|---|---|
| Yes: | 11 |
| No: | 2 |

The tabulated results suggest that the test composition containing Sennosides and Glucomannan (Composition B) was well-tolerated with 8-of-13 reporting no side-effects, with 5 reports of side-effects. In contrast, only 4-of-11 subjects reported no side-effects with Composition A, with 12 reports of side-effects (note: some subjects reported multiple side-effects categories). Of particular interest were the “Time for effect” results where 9-of-13 subjects reported experiencing effects after between 9-12 hours with Composition B, with only 4 subjects experiencing effects earlier. This is about twice as long as the expected time of 4-5 hours for the effects of Anthranoid laxatives alone. It is to be appreciated that the longer duration of laxative action of Composition B is advantageous for increased efficacy. Alternatively, it is also appreciated that the dosage of Sennosides in Composition B may be lowered to achieve similar duration and/or effect as Anthranoid laxatives alone (Composition A).

With the subjects consuming Composition A, just as many subjects experienced effects between 9-12 hours as did earlier than 9 hours (4 subjects for each). Also, Composition B appears to have a greater effect for increasing volume.

Of the 7 subjects that were given both Composition A and Composition B, 5 reported that composition B was a more effective laxative, 1 reported no difference, and 1 reported the composition A was more effective.

### Example 2

### Laxative Composition

According to an aspect, the laxative composition of the present invention comprises the following:

Gastrointestinal & Digestive Support
Glucomannan powder (as Amorphophallus konjac)
Senna extract (as Cassia angustifolia)
Cascara sagrada extract (as Rhamnus purshiana)
Psyllium powder (as Plantago ovata)
Burdock extract (as Arctium lappa)
Red clover extract (as Trifolium pratense)
Guar gum (as Cyamopsis tetragonoloba)
Slippery elm powder (as Ulmus rubra)
Ginger extract (as Zingiber officinale)
Rhubarb extract (as Rheum officinale)
Fennel extract (as Foeniculum vulgare)
Alfalfa powder (as Medicago sativa)
Xanthan gum
Intestinal Pre-biotic Support
Inulin (as Cichorium intybus)
Oligofructose
Methylcellulose
Liver Health Support
Milk thistle extract (as Silybum marianum)
Turmeric powder (as Curcuma longa)
Picrorhiza kurrooa extract
Artichoke extract (as Cynara scolymus)
Betaine HCl
Hyssopus officinalis extract
Flushing Support
Parsley extract (as Petroselinum crispum)
Dandelion extract (as Taraxacum officinale)
Licorice extract (as Glycyrrhiza glabra)
Uva ursi extract (as Arctostaphylos uva-ursi)
Cranberry extract (as Vaccinium macrocarpon)
Chamomile extract (as Matricaria recutita)
Marshmallow extract (as Althaea officinalis)
Opuntia ficus-indica powder
European ash extract (as Fraxinus excelsior)
Hibiscus powder (as Hibiscus sabdariffa)
Wellness Support
Alpha lipoic acid
L-cysteine HCl
Blueberry extract (as Vaccinium angustifolium)

### Extensions and Alternatives

In the foregoing specification, the invention has been described with a specific embodiment thereof; however, it will be evident that various modifications and changes may be made thereto without departing from the broader spirit and scope of the invention.

What is claimed:

1. A laxative composition comprising at least one Anthranoid laxative and at least one soluble fiber.

2. The laxative composition of claim 1, wherein the Anthranoid laxative is selected from the group consisting of Senna, Cascara and Rhubarb.

3. The laxative composition of claim 1, wherein the soluble fiber is selected from the group consisting of Glucomannan, Guar gum, Xanthan gum, and Hydroxypropylmethyl cellulose.

4. The laxative composition of claim 1 further comprising an agent that promotes healthy intestinal function.

5. The laxative composition of claim 4 wherein the agent that promotes healthy intestinal function is inulin.

6. The laxative composition of claim 1 further comprising an agent that promotes healthy liver function.

7. The laxative composition of claim 6 wherein the agent that promotes healthy liver function is an extract of Silybum marianum.

8. The laxative composition of claim 6 wherein the agent that promotes healthy liver function is an extract of Taraxacum officinale.

9. The laxative composition of claim 1 further comprising an agent that reduces carbohydrate absorption.

10. The laxative composition of claim 9 wherein the agent that reduces carbohydrate absorption is an extract of Morus alba.

11. The laxative composition of claim 9 wherein the agent that reduces carbohydrate absorption is an extract of Phaseolus vulgaris.

12. The laxative composition of claim 1 further comprising an antioxidant.

13. A method for reducing the negative effects of Anthranoid laxatives comprising providing a laxative composition comprising at least one Anthranoid laxative and at least one soluble fiber.

14. The method of claim 13, wherein the Anthranoid laxative is selected from the group consisting of Senna, Cascara and Rhubarb.

15. The method of claim 13, wherein the soluble fiber is selected from the group consisting of Glucomannan, Guar gum, Xanthan gum, and Hydroxypropylmethyl cellulose.

16. A method for increasing at least one of efficacy and duration of effect of Anthranoid laxatives comprising providing a laxative composition comprising at least one Anthranoid laxative and at least one soluble fiber.

17. The method of claim 16, wherein the Anthranoid laxative is selected from the group consisting of Senna, Cascara and Rhubarb.

18. The method of claim 16, wherein the soluble fiber is selected from the group consisting of Glucomannan, Guar gum, Xanthan gum, and Hydroxypropylmethyl cellulose.
